(19) United States
(12) Patent Application Publication
(10) Pub. No.: US 2011/0284014 A1
(43) Pub. Date: Nov. 24, 2011

(54) Medical Devices That Include Removable Magnet Units and Related Methods

(75) Inventors: Jeffrey A. Cadeddu, Dallas, TX (US); Daniel J. Scott, Dallas, TX (US); Raul Fernandez, Arlington, TX (US); Heather Beardsley, Arlington, TX (US); Richard A. Bergs, Grand Prairie, TX (US)

(73) Assignee: The Board of Regents of the University of Texas System

(21) Appl. No.: 12/783,430

(22) Filed: May 19, 2010

(51) Int. Cl. A61B 19/00

(52) U.S. Cl. 128/899

(57) ABSTRACT

Medical devices and methods for magnetically positioning a device within a body cavity of a patient, including a removable magnet unit that can be reused across procedures.

### BACKGROUND

1. Field of the Invention

The present invention relates generally to medical devices, systems, and methods, and, more particularly, but not by way of limitation, to medical devices, systems, and methods that include an internal platform configured to be positioned within a body cavity, where the platform includes a reusable magnet unit that is removably couplable to a base.

2. Description of Related Art

For illustration, the background is described with respect to medical procedures (e.g., surgical procedures), which can include laparoscopy, transmural surgery, and endoluminal surgery, including, for example, natural orifice transluminal endoscopic surgery (NOTES), single-incision laparoscopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, systems, and methods, and each is incorporated by reference in its entirety: (1) International Application No. PCT/US2009/063987, filed on Nov. 11, 2009; (2) U.S. patent application Ser. No. 10/024,636, filed Dec. 14, 2001, and published as Pub. No. US 2003/0114731; (3) U.S. patent application Ser. No. 10/999,396, filed Nov. 30, 2004, published as Pub. No. US 2005/0165449 and issued as U.S. Pat. No. 7,429,259; (4) U.S. patent application Ser. No. 11/741,731, filed Apr. 28, 2007, published as Pub. No. US 2007/0255273 and issued as U.S. Pat. No. 7,691,103; (5) U.S. patent application Ser. No. 12/146,953, filed Jun. 26, 2008, and published as Pub. No. US 2008/0269779; and (6) U.S. patent application Ser. No. 12/755,312, filed on Apr. 6, 2010.

### SUMMARY

Medical devices and methods that include an internal platform having a removable magnet unit that is couplable to a base. Some embodiments of the present medical devices comprise an internal platform configured to be inserted within a body cavity (e.g., of a patient), the internal platform having a base and a removable magnet unit couplable to the base, the removable magnet unit having multiple magnets positioned in a biocompatible housing such that the multiple magnets have no exposed surface. In certain embodiments, the medical devices may also include an external unit configured to be positioned outside the body cavity and be magnetically coupled to the internal platform. In some embodiments, the internal platform, the external unit, or both, may be sterile.

Some embodiments of the present medical devices comprise an internal platform configured to be inserted within a body cavity of a patient, the internal platform having a base and a removable magnet unit couplable to the base, the removable magnet unit having multiple magnets encased in a biocompatible housing. In certain embodiments, such medical devices may also include an external unit configured to be positioned outside the body cavity and be magnetically coupled to the internal platform. In some embodiments, the internal platform, the external unit, or both, may be sterile.

Some embodiments of the present methods comprise performing a first procedure that includes positioning an internal platform in a body cavity, the internal platform comprising a base and a removable magnet unit coupled to the base; and magnetically coupling an external unit across tissue to the internal platform. In some embodiments, the method also includes performing a second procedure using another internal platform that includes another base coupled to the removable magnet unit. In some embodiments, the internal platform, the external unit, or both, may be sterile for the first procedure. The removable magnet unit, along with the base of the other internal platform, may also be sterile for the second procedure.

The present internal platforms may be characterized as defining a longitudinal axis along their respective lengths and having a maximum transverse perimeter, which is defined by the smallest circle or rectangle that can circumscribe the largest cross-section of the platform taken perpendicular to the longitudinal axis. In some embodiments, the maximum transverse perimeter of the present internal platforms is less than 7 inches. In some embodiments, the area circumscribed by the maximum transverse perimeter is less than 3.2 square inches.

Any embodiment of any of the present medical devices, systems, and methods can consist of or consist essentially of—rather than comprise/include/contain/have—any of the described elements and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 depicts a graphical representation of one of the present medical devices positioned within a body cavity of a patient and magnetically coupled to a positioning apparatus that is located outside the cavity.

FIG. 2 is a perspective view of one embodiment of the present internal platforms that includes a removable magnet unit.

FIG. 3A is a cross-sectional view of the removable magnet unit shown in FIG. 2 and taken along the arrows shown in FIG. 2.

FIG. 3B is a cross-sectional view of the internal platform shown in FIG. 2 and taken along the arrows shown in FIG. 2.

FIGS. 4 and 5 are exploded perspective views of the internal platform shown in FIG. 2.

FIG. 6 is a side view of one of the bases of the present internal platforms.

FIG. 7 is a partial cross-sectional view of another of the bases of the present internal platforms.

FIG. 8 is a side view of still another of the bases of the present internal platforms.

FIG. 9 is an exploded perspective view another embodiment of the present internal platform.

FIG. 10 is a side view of one of the present medical devices that includes an external unit and an internal platform having a removable magnet unit.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The term “substantially” is defined as being largely but not necessarily wholly what is specified, as understood by a person of ordinary skill in the art. For example, in any of the present embodiments, the term “substantially” may be substituted with “within [a percentage] of what is specified, where the percentage includes any of 5, 10, and/or 15 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a medical device or method that “comprises,” “has,” “includes” or “contains” one or more elements or steps possesses those one or more elements or steps, but is not limited to possessing only those one or more elements or steps. Likewise, an element of a medical device that “comprises,” “has,” “includes” or “contains” one or more features possesses those one or more features, but is not limited to possessing only those one or more features. For example, an internal platform that comprises a base and a removable magnet unit couplable to the base includes the specified features but is not limited to having only those features. Such an internal platform could also include, for example, an arm coupled to the base.

Further, a device or structure that is configured in a certain way is configured in at least that way, but it can also be configured in other ways than those specifically described.

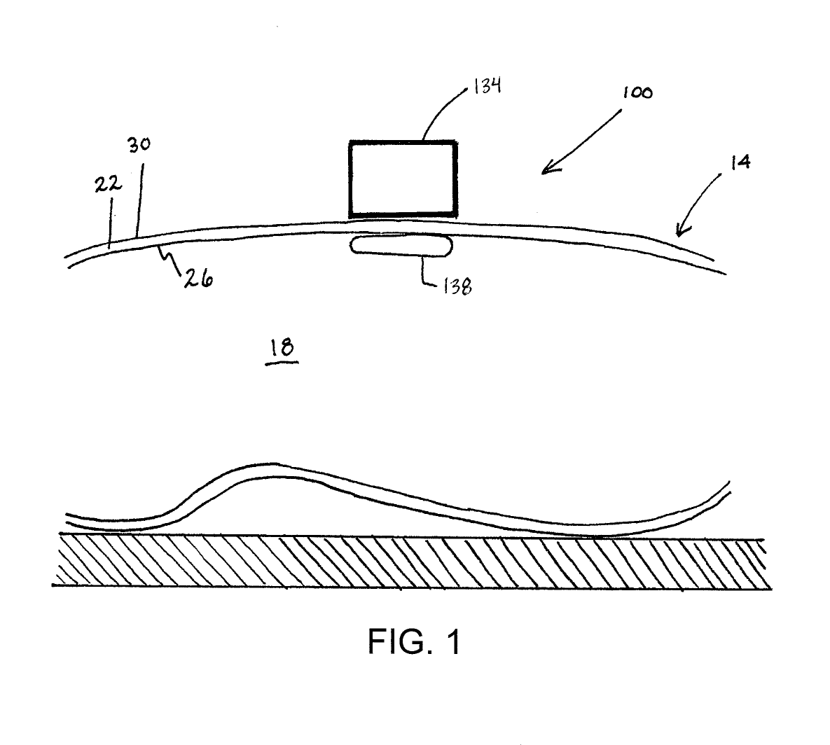

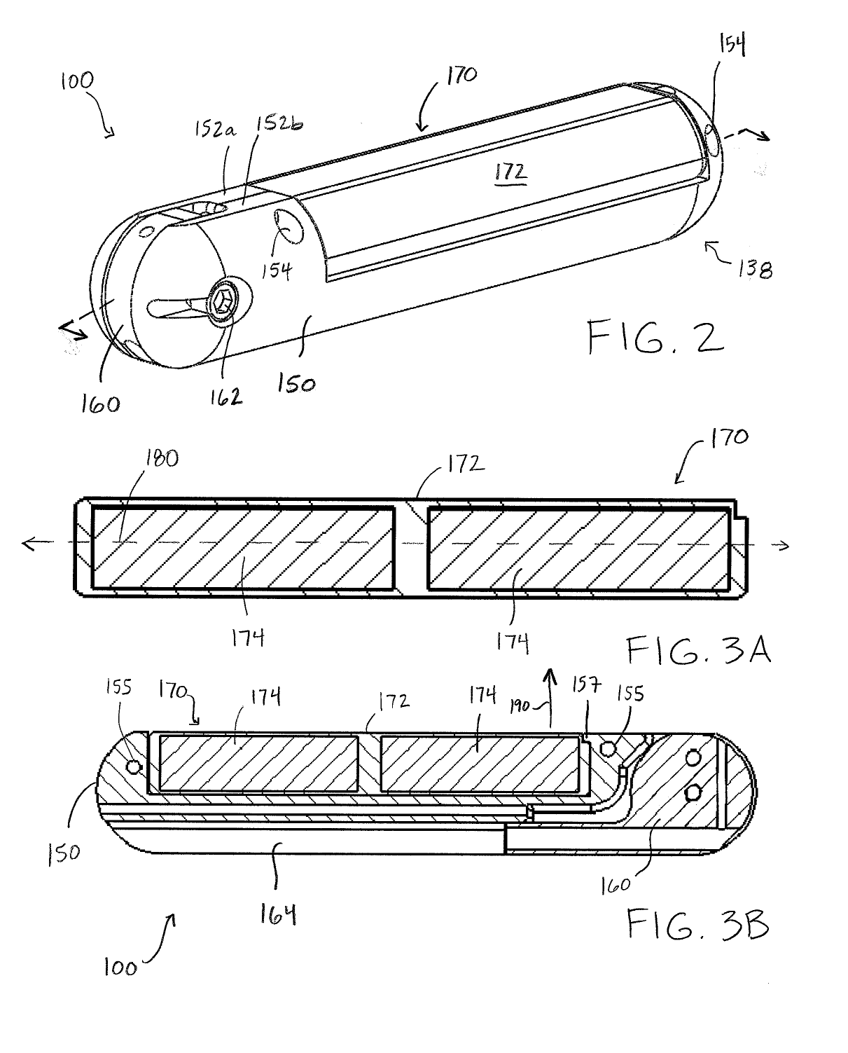

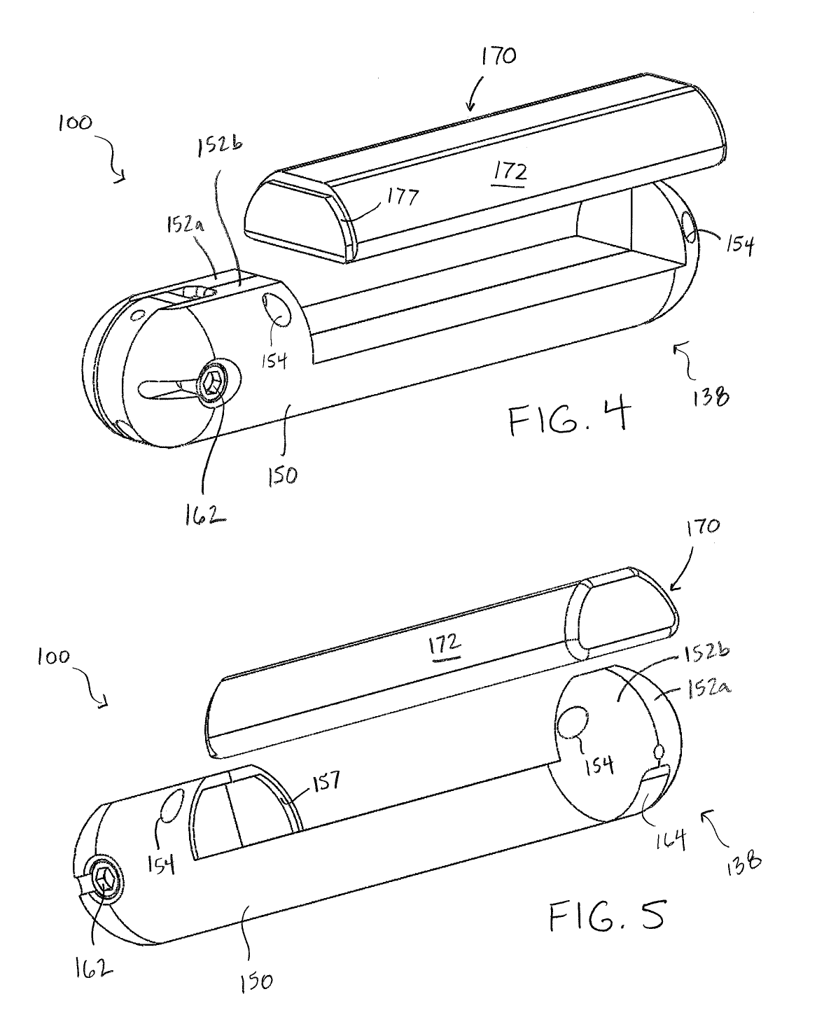

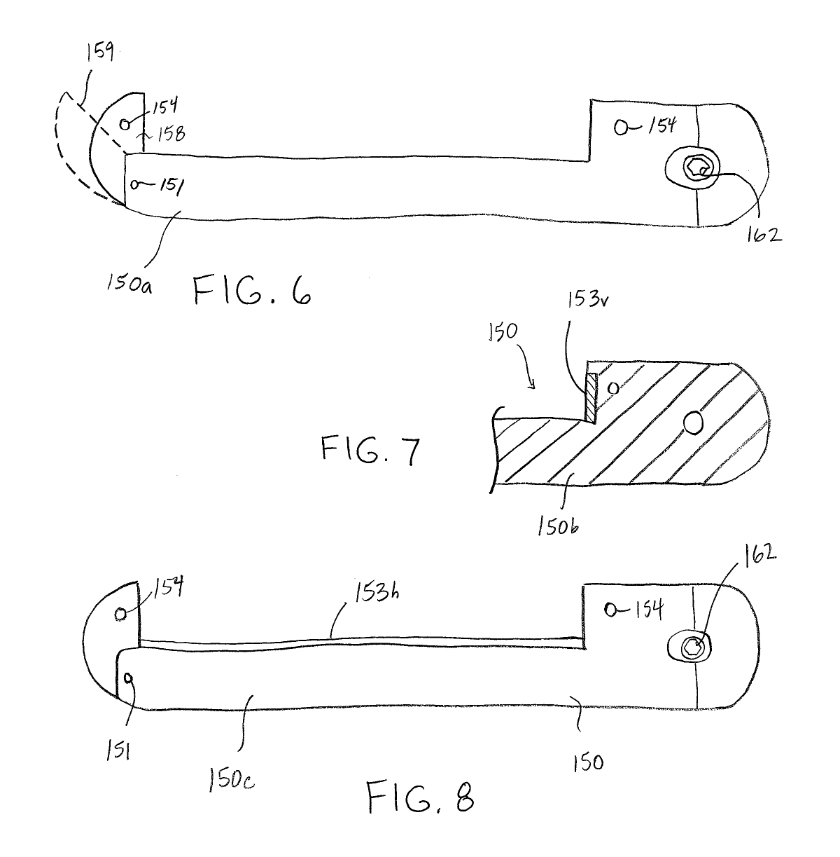

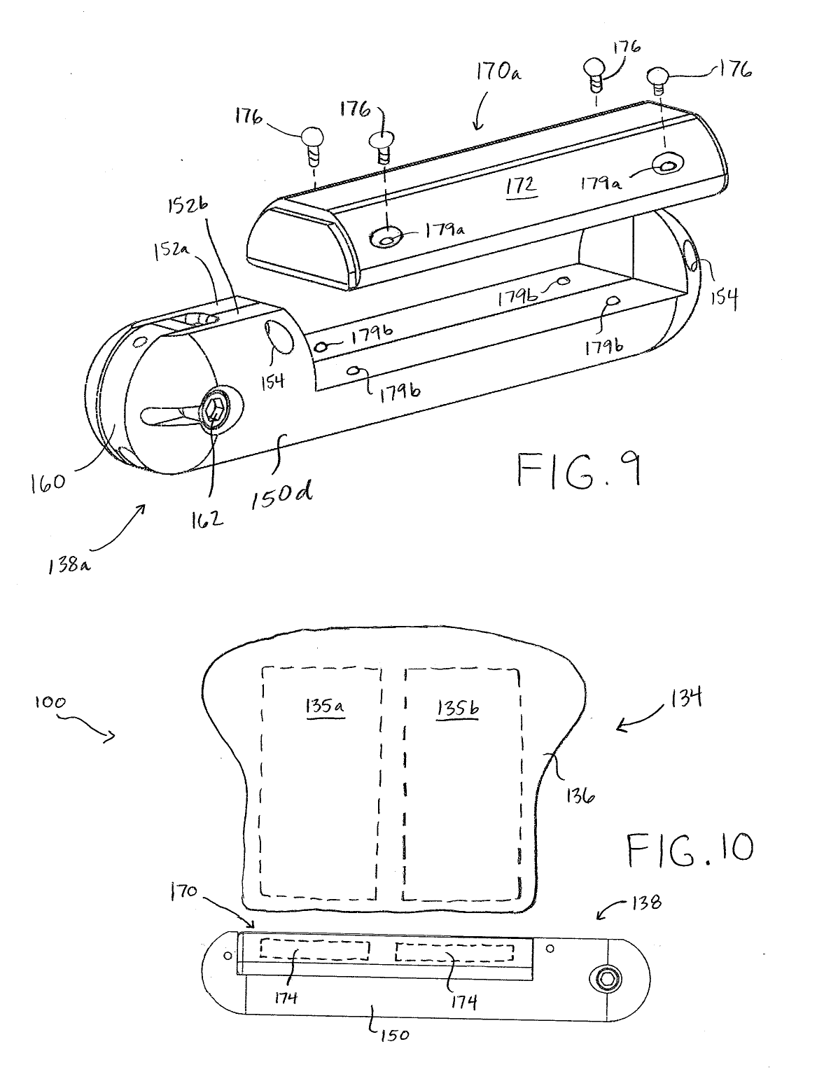

Referring now to the drawings, shown in FIG. 1 by reference numeral 100 is a graphical representation of one embodiment of the present medical devices, which can be used in medical procedures. Details of the components of medical device 100 are provided in, for example, FIGS. 2-5 and 10.

Medical device 100 is shown in conjunction with a body 14 (which may be a patient), and more particularly in FIG. 1 is shown relative to a longitudinal cross-sectional view of the ventral cavity 18 of a human patient 14. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIG. 1, various embodiments of the present invention (including the version of medical device 100 shown in the figures) can also be used with other animals, such as in veterinary medical procedures.

Further, although medical device 100 is depicted relative to ventral cavity 18, medical device 100 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIG. 1, medical device 100 comprises an external unit 134 and a internal platform 138; the external unit is configured to be positioned outside a body cavity of a patient and magnetically position the internal platform within the body cavity. Embodiments of the present “medical devices” or “systems” can include an internal platform (like internal platform 138) and, in a subset of embodiments, both an internal platform and an exterior unit (like exterior unit 134) that is configured to be magnetically coupled to the internal platform.

As shown, external unit 134 can be positioned outside the cavity 18 near, adjacent to, and/or in contact with the exterior surface 30 of the patient 14. Internal platform 138 is positionable (can be positioned), and is shown positioned, within the cavity 18 of the patient 14 and near, adjacent to, and/or in contact with the interior surface 26 of wall 22. Internal platform 138 can be inserted or introduced into the cavity 18 in any suitable fashion. For example, the internal platform 138 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with external unit 134 once external unit 134 has been magnetically coupled to internal platform 138. If the cavity 18 is pressurized, internal platform 138 can be inserted or introduced into the cavity 18 before or after the cavity 18 is pressurized. Additionally, some embodiments of medical device 100 include a version of internal platform 138 that has a tether (not shown) coupled to and extending away from the internal platform 138.

External unit 134 and internal platform 138 can be configured to be magnetically couplable to one another such that internal platform 138 can be positioned or moved within the cavity 18 by positioning or moving external unit 134 outside the cavity 18. “Magnetically couplable” means capable of magnetically interacting so as to achieve a physical result without a direct physical connection. Examples of physical results are causing internal platform 138 to move within the cavity 18 by moving external unit 134 outside the cavity 18, and causing internal platform 138 to remain in a position within the cavity 18 or in contact with the interior surface 26 of wall 22 by holding external unit 134 in a corresponding position outside the cavity 18 or in contact with the exterior surface 30 of wall 22. Magnetic coupling can be achieved by configuring external unit 134 and internal platform 138 to cause a sufficient magnetic attractive force between them.

For example, external unit 134 can comprise one or more magnets (e.g., permanent magnets, electromagnets, or the like) and internal platform 138 can comprise a ferromagnetic material. In some embodiments, external unit 134 can comprise one or more magnets, and internal platform 138 can comprise a ferromagnetic material, such that external unit 134 attracts internal platform 138 and internal platform 138 is attracted to external unit 134. In other embodiments, both external unit 134 and internal platform 138 can comprise one or more magnets such that external unit 134 and internal platform 138 attract each other.

The configuration of external unit 134 and internal platform 138 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when external unit 134 and internal platform 138 are magnetically coupled as shown, with each contacting a respective surface 26 or 30 of wall 22, the magnetic force between them can compress wall 22 to some degree such that wall 22 exerts a spring or expansive force against external unit 134 and internal platform 138, and such that any movement of external unit 134 and internal platform 138 requires an adjacent portion of wall 22 to be similarly compressed. As discussed further below, external unit 134 and internal platform 138 can be configured to overcome such an impeding force to the movement of internal platform 138 with external unit 134. Another force that the magnetic attractive force between the two may have to overcome is any friction that exists between either and the surface, if any, that it contacts during a procedure (such as external unit 134 contacting a patient's skin)

In some embodiments, internal platform 138 can be inserted into cavity 18 through an access port having a suitable internal diameter. Such access ports includes those created using a conventional laparoscopic trocar, gel ports, those created by incision (e.g., abdominal incision), and natural orifices. Internal platform 138 can be pushed through the access port with any elongated instrument such as, for example, a surgical instrument such as a laparoscopic grasper or a flexible endoscope.

In some embodiments, when internal platform 138 is disposed within cavity 18, internal platform 138 can be magnetically coupled to external unit 134. This can serve several purposes including, for example, to permit a user to move internal platform 138 within cavity 18 by moving external unit 134 outside cavity 18. The magnetic coupling between the two can be affected by a number of factors, including the distance between them. For example, the magnetic attractive force between internal platform 138 and external unit 134 increases as the distance between them decreases. As a result, in some embodiments, the magnetic coupling can be facilitated by temporarily compressing the tissue (e.g., the abdominal wall) separating them. For example, after internal platform 138 has been inserted into cavity 18, a user (such as a surgeon) can push down on external unit 134 (and wall 22) and into cavity 18 until external unit 134 and internal platform 138 magnetically couple.

In FIG. 1, external unit 134 and internal platform 138 are shown at a coupling distance from one another and magnetically coupled to one another such that internal platform 138 can be moved within the cavity 18 by moving external unit 134 outside the outside wall 22. The “coupling distance” between two structures (e.g., external unit 134 and internal platform 138) is defined as a distance between the closest portions of the structures at which the magnetic attractive force between them is great enough to permit them to function as desired for a given application.

The “maximum coupling distance” between two structures (e.g., external unit 134 and internal platform 138) is defined as the greatest distance between the closest portions of the structures at which the magnetic attractive force between them is great enough to permit them to function as desired for a given application. Factors such as the thickness and composition of the matter (e.g., human tissue) separating them can affect the coupling distance and the maximum coupling distance for a given application. For example, in the embodiment shown in FIG. 1, the maximum coupling distance between external unit 134 and internal platform 138 is the maximum distance between them at which the magnetic attractive force is still strong enough to overcome the weight of internal platform 138, the force caused by compression of wall 22, the frictional forces caused by contact with wall 22, and any other forces necessary to permit internal platform 138 to be moved within cavity 18 by moving external unit 134 outside wall 22. In some embodiments, external unit 134 and internal platform 138 can be configured to be magnetically couplable such that when within a certain coupling distance of one another the magnetic attractive force between them is strong enough to support the weight of internal platform 138 in a fixed position and hold internal platform 138 in contact with the interior surface 26 of wall 22, but not strong enough to permit internal platform 138 to be moved within the cavity 18 by moving external unit 134 outside wall 22.

In some embodiments, external unit 134 and internal platform 138 can be configured to have a minimum magnetic attractive force at a certain distance. For example, in some embodiments, external unit 134 and internal platform 138 can be configured such that at a distance of 50 millimeters between the closest portions of external unit 134 and internal platform 138, the magnetic attractive force between external unit 134 and internal platform 138 is at least about: 20 grams, 25 grams, 30 grams, 35 grams, 40 grams, or 45 grams. In some embodiments, external unit 134 and internal platform 138 can be configured such that at a distance of about 30 millimeters between the closest portions of external unit 134 and internal platform 138, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, external unit 134 and internal platform 138 can be configured such that at a distance of about 15 millimeters between the closest portions of external unit 134 and internal platform 138, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, external unit 134 and internal platform 138 can be configured such that at a distance of about 10 millimeters between the closest portions of external unit 134 and internal platform 138, the magnetic attractive force between them is at least about: 2000 grams, 2200 grams, 2400 grams, 2600 grams, 2800 grams, 3000 grams, 3200 grams, 3400 grams, 3600 grams, 3800 grams, or 4000 grams. These distances may be coupling distances or maximum coupling distances for some embodiments.

FIGS. 2-9 show different embodiments of the present medical devices featuring different embodiments of the present internal platforms. These figures show details of internal platforms not illustrated in FIG. 1. As shown in FIGS. 2-5, internal platform 138 of medical device 100 has base 150 and removable magnet unit 170 that is couplable to (and, in the depicted embodiment, coupled to) the base. Removable magnet unit 170 includes housing 172 and multiple magnets (in this embodiment, two) 174 positioned in the housing. More specifically, the magnets are positioned in the housing such that the magnets have no exposed surface, meaning no surface of the magnets can be contacted from outside the housing without penetrating some of the feature of the unit, such as the housing. Removable magnet unit 170 may be characterized as having multiple magnets 174 encased in, or embedded in, housing 172.

Base 150 includes two halves 152a and 152b that are coupled together. In this embodiment, fasteners (not shown) are positioned through coupling openings 155, which are accessible through recesses 154. Internal platform 138 also includes an arm 160 that is coupled (rotatably or pivotally coupled, in this embodiment) to base 150, and that fits substantially or completely within slot 164 of base 150 in its collapsed position. Although not shown, internal platform 138 can also include a tool (such as a cautery device or a camera) coupled to arm 160. Arm 160 can be actuated in any suitable manner, such as through rotation of hex opening 162, which may be part of a nut or the like that is directly connected to arm 160 such that rotation of hex opening 162 translates directly into rotation of arm 160 for the purpose of orienting arm 160 in a deployed position. An arm actuation tool (not shown) that is configured to interface with hex opening 162 may be included as part of medical device 100.

As shown in FIG. 3A, removable magnet unit 170 defines longitudinal axis 180, which is an axis that is oriented lengthwise through the unit. Base 150 includes retention shoulder 157, and removable magnet unit 170 includes a retention member 177. Base 150 and removable magnet unit 170 are configured such that when the base and the removable magnet unit are coupled together, the retention shoulder interferes with movement of the retention member in at least one direction that is perpendicular to the longitudinal axis, such as direction 190 shown in FIG. 3A.

Another embodiment of the bases of the present internal platforms is shown in FIG. 6. Base 150a, which can be used instead of base 150 with internal platform 138 of medical device 100, includes end member 158 that is configured to move between an open position (shown in phantom as position 159) and the closed position shown in unbroken lines. Open position 159 facilitates the coupling and decoupling of removable magnet unit 170 (not shown in FIG. 6) to and from base 150a. The closed position of end member 158 facilitates retention of removable magnet unit 170 by base 150a. End member 158 can be pivotally (or rotatably) coupled to the balance of base 150a with pin 151, such that end member 158 is capable of being manually or automatically rotated about the axis (not shown) defined by pin 151. End member 158 can be biased to the closed position using any suitable structure(s), such as, for example, a spring or a magnet. In other embodiments, end member 158 is not biased, and may be held in the closed position using any suitable structure, such as a detent. Although not visible in FIG. 6, base member 150a may, in some embodiments, include the retention shoulder of base 150.

Another embodiment of the bases of the present internal platforms is shown in FIG. 7. Base 150b, which can be used instead of base 150 or 150a with internal platform 138 of medical device 100, includes magnetic material 153v that is coupled to the balance of the base and configured to magnetically couple the removable magnet unit to the base. Magnetic material 153v is oriented to contact a forward end surface of a removable magnet unit (not shown); in such embodiments, the unit may not have a retention member like retention member 177. As a result of the orientation of magnetic material 153v, it lies in a plane (not shown) that is oriented perpendicular to the longitudinal axis defined by the removable magnet unit when the base and unit are coupled together. Magnetic material 153v, which may be ferromagnetic material (such as carbon steel), may be coupled to the balance of base 150b in any suitable fashion, such as through an adhesive, a slotted connection, a friction fit, embedding, or the like. Although not shown in FIG. 7, base 150b can include end member 158 in some embodiments.

Another embodiment of the bases of the present internal platforms is shown in FIG. 8. Base 150c, which can be used instead of base 150, 150a, or 150b with internal platform 138 of medical device 100, includes magnetic material 153h that is coupled to the balance of the base and configured to magnetically couple the removable magnet unit to the base. Magnetic material 153h is oriented to contact a bottom surface of a removable magnet unit (not shown). As a result, it lies in a plane (not shown) that is oriented parallel to the longitudinal axis defined by the removable magnet unit, when the base and unit are coupled together. As shown, base 150c can include end member 158 in some embodiments. In other embodiments, end member 158 is not included. Some embodiments of base 150c include the retention shoulder of base 150, and others do not. Magnetic material 153h, which may be ferromagnetic material (such as carbon steel), may be coupled to the balance of base 150c in any suitable fashion, such as through an adhesive, a slotted connection, a friction fit, embedding, or the like. In other embodiments, the base includes both magnetic materials 153v and 153h, and or those materials may be unitary.

Another embodiment of the present internal platforms is shown in FIG. 9. Internal platform 138a, which can be used instead of internal platform 138 of medical device 100, includes removable magnet unit 170a and base 150d, which are configured to be coupled together with fasteners, which may be threaded fasteners 176 (e.g., screws). Removable magnet unit 170a is the same as removable magnet unit 170, except that unit 170a includes multiple openings 179a configured to accept fasteners, such as threaded fasteners 176, though openings 179a need not be threaded. Base 150d is the same as base 150, except that base 150d includes multiple openings 179b configured to accept fasteners, such as threaded fasteners 176. In this embodiment, openings 179b are threaded.

Some embodiments of the present medical devices and systems also include an external unit. For example, as shown in FIG. 1 and with more particularity in FIG. 10, medical device 100 may also include external unit 134, which is configured to be placed outside a body cavity and magnetically coupled to internal platform 138 through a tissue. In the depicted embodiment, external unit 134 comprises first magnet 135a and second magnet 135b. These magnets are positioned in (e.g., embedded or encased in) housing 136, which is similar in nature to the housing of removable magnet unit 170. First magnet 135a is configured to be magnetically coupled to one of magnets 174 of unit 170 and second magnet 135b is configured to be magnetically coupled to another of magnets 174 (and, in this embodiment, the other magnet 174).

Some embodiments of the present removable magnet units may be re-used. For example, after a given procedure, the internal platform may be removed from the body cavity, the base may be disposed of, and the removable magnet unit may be cleaned, sterilized, and stored for later use with another disposable base. Thus, some embodiments of the present methods include performing a procedure using one of the present internal platforms, cleaning and sterilizing the removable magnet unit of the platform, and re-using the unit with another internal platform in another procedure (and, more specifically, with another the base of another internal platform in another procedure). Suitable medical procedures include surgical procedures such as, for example, natural orifice transluminal endoscopic surgery (NOTES), single-incision laparoscopic surgery (SILS), single-port laparoscopy (SLP), and others.

The internal platform and, in some cases, the external unit of a given one of the present medical devices or systems may be placed in a sterile, sealed package that can be removed before a procedure. The platforms themselves, as well as the external units, may be sterilized using any suitable technique. In addition, in embodiments of the present methods, a given internal platform and/or external unit may be placed or wrapped in a sterile barrier (e.g., a sheet, a paper or a film) after being taken out of its package and before being used in a procedure.

The materials from which the present bases and/or housings can be made of include those that are biocompatible, including biocompatible plastics, metals, composites, alloys, and the like. The present internal platforms and external units can be made using any suitable techniques, including molding (e.g., injection molding), conventional subtractive methods such as milling or turning, or additive methods such as those used for rapid prototyping. Examples of suitable magnets for use in the present removable magnet units and external units include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929.

The various illustrative embodiments of systems, medical devices, and methods described in this disclosure are not intended to be limited to the particular forms disclosed. Rather, they include all modifications and alternatives falling within the scope of the claims. For example, while base 150 includes retention shoulder 158, base 150 could include an alternative structure or structures for retaining the removable magnet unit, such as a slot or slots; and while removable magnet unit 170 includes a retention member 177 configured to interfere with retention shoulder 158 when base 150 and unit 170 are coupled together, for the purpose of keeping them coupled together during a procedure, unit 170 could include an alternative structure or structures for being couplable to base 150, such as a projection or projections configured to mate with the slot or slots of the base.

The claims are not intended to include, and should not be interpreted to include, means-plus- or step-plus-function limitations, unless such a limitation is explicitly recited in a given claim using the phrase(s) “means for” or “step for,” respectively.

1. A medical device comprising:
an internal platform configured to be inserted within a body cavity of a patient, the internal platform having a base and a removable magnet unit couplable to the base, the removable magnet unit having multiple magnets positioned in a biocompatible housing such that the multiple magnets have no exposed surface.

2. The medical device of claim 1, where the removable magnet unit defines a longitudinal axis, the base includes a retention shoulder and the removable magnet unit includes a retention member, and the base and the removable magnet unit are configured such that when the base and the removable magnet unit are coupled together, the retention shoulder interferes with movement of the retention member in at least one direction perpendicular to the longitudinal axis.

3. The medical device of any of claims 1-2, where the base also includes an end member configured to move between an open position and a closed position, and where the open position facilitates coupling and decoupling the removable magnet unit to and from the base and the closed position facilitates retention of the removable magnet unit by the base.

4. The medical device of claim 1, where the base includes magnetic material configured to magnetically couple the removable magnet unit to the base.

5. The medical device of claim 1, where the base and the removable magnet unit are configured to be coupled together with fasteners.

6. The medical device of claim 5, where the base and the removable magnet unit are configured to be threadably coupled together with fasteners.

7. The medical device of any of claims 1-5, further comprising an external unit configured to be placed outside the body cavity and magnetically coupled to the internal platform through a tissue.

8. The medical device of claim 7, where the external unit comprises a first magnet and a second magnet, the first magnet being configured to be magnetically coupled to one of the multiple magnets and the second magnet being configured to be magnetically coupled to another of the multiple magnets.

9. A medical device comprising:
an internal platform configured to be inserted within a body cavity of a patient, the internal platform having a base and a removable magnet unit couplable to the base, the removable magnet unit having multiple magnets encased in a biocompatible housing.

10. The medical device of claim 9, where the removable magnet unit defines a longitudinal axis, the base includes a retention shoulder and the removable magnet unit includes a retention member, and the base and the removable magnet unit are configured such that when the base and the removable magnet unit are coupled together, the retention shoulder interferes with movement of the retention member in at least one direction perpendicular to the longitudinal axis.

11. The medical device of any of claims 9-10, where the base also includes an end member configured to move between an open position and a closed position, and where the open position facilitates coupling and decoupling the removable magnet unit to and from the base and the closed position facilitates retention of the removable magnet unit by the base.

12. The medical device of claim 9, where the base includes magnetic material configured to magnetically couple the removable magnet unit to the base.

13. The medical device of claim 9, where the base and the removable magnet unit are configured to be coupled together with fasteners.

14. The medical device of claim 13, where the base and the removable magnet unit are configured to be threadably coupled together with fasteners.

15. The medical device of any of claims 9-14, further comprising an external unit configured to be placed outside the body cavity and magnetically coupled to the internal platform through a tissue.

16. The medical device of claim 15, where the external unit comprises a first magnet and a second magnet, the first magnet being configured to be magnetically coupled to one of the multiple magnets and the second magnet being configured to be magnetically coupled to another of the multiple magnets.

17. A method comprising:
performing a first procedure including:
positioning an internal platform in a body cavity, the internal platform comprising a base and a removable magnet unit coupled to the base;
magnetically coupling an external unit across tissue to the internal platform;
performing a second procedure using another internal platform that includes another base coupled to the removable magnet unit.

18. The method of claim 17, where the removable magnet unit has multiple magnets.

19. The method of claim 18, where the external unit comprises a first magnet and a second magnet, the first magnet being configured to be magnetically coupled to one of the multiple magnets and the second magnet being configured to be magnetically coupled to another of the multiple magnets.
